(19) United States
(12) Plant Patent Application Publication
(10) Pub. No.: US 2018/0242503 P1
(43) Pub. Date: Aug. 23, 2018

(54) CALIBRACHOA PLANT NAMED 'DUEALKONEO'

(50) ;

(71) Applicant: DUMMEN GROUP B.V., De LIER (NL)

(72) Inventor: Arjan Koot, Oeffelt (NL)

(73) Assignee: DUMMEN GROUP B.V., De LIER (NL)

(21) Appl. No.: 15/530,756

(22) Filed: Feb. 22, 2017

(51) Int. Cl. A01H 6/82

(52) U.S. Cl. PLT/413

(57) ABSTRACT

A new and distinct cultivar of Calibrachoa plant named ‘Duealkoneo’, characterized by its compact, semi-upright to outwardly spreading, trailing and decumbent plant habit; freely branching habit; early and freely flowering habit; flowers with red purple-colored petals with dark purple-colored centers; and good garden performance.

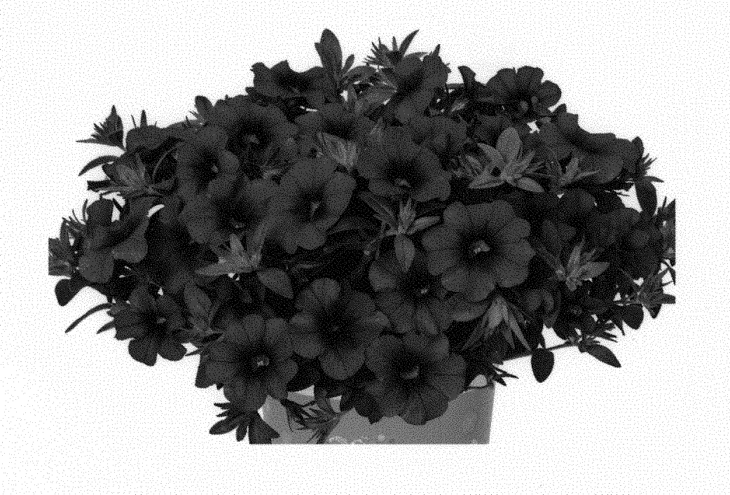

Botanical designation: Calibrachoa sp.

Cultivar denomination: ‘DUEALKONEO’.

### BACKGROUND OF THE INVENTION

The present invention relates to a new and distinct cultivar of Calibrachoa plant, botanically known as Calibrachoa sp. and hereinafter referred to by the name ‘Duealkoneo’.

The new Calibrachoa plant is a product of a planned breeding program conducted by the Inventor in Rheinberg, Germany. The objective of the breeding program is to create new compact Calibrachoa plants with unique and attractive flowers.

The new Calibrachoa plant originated from a cross-pollination made by the Inventor in July, 2014 in Rheinberg, Germany of a proprietary selection of Calibrachoa sp. identified as code number AA12-002806-002, not patented, as the female, or seed, parent with a proprietary selection of Calibrachoa sp. identified as code number AA13-003709-002, not patented, as the male, or pollen, parent. The new Calibrachoa plant was discovered and selected by the Inventor as a single flowering plant from within the progeny of the stated cross-pollination in a controlled greenhouse environment in Rheinberg, Germany in May, 2016.

Asexual reproduction of the new Calibrachoa plant by vegetative terminal cuttings in a controlled greenhouse environment in Rheinberg, Germany since June, 2016 has shown that the unique features of this new Calibrachoa plant are stable and reproduced true to type in successive generations.

### SUMMARY OF THE INVENTION

Plants of the new Calibrachoa have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Duealkoneo’. These characteristics in combination distinguish ‘Duealkoneo’ as a new and distinct Calibrachoa plant:
1. Compact, semi-upright to outwardly spreading, trailing and decumbent plant habit.
2. Freely branching habit.
3. Early and freely flowering habit.
4. Flowers with red purple-colored petals with dark purple-colored centers.
5. Good garden performance.

The new Calibrachoa can be compared to plants of the female parent selection. Plants of the new Calibrachoa differ primarily from plants of the female parent selection in plant habit as plants of the new Calibrachoa are more compact than plants of the female parent selection. In addition, plants of the new Calibrachoa have larger flowers than plants of the female parent selection.

The new Calibrachoa can be compared to plants of the male parent selection. Plants of the new Calibrachoa differ primarily from plants of the male parent selection in flowering habit as plants of the new Calibrachoa flower earlier than plants of the male parent selection. In addition, plants of the new Calibrachoa are more freely branching than plants of the male parent selection.

Plants of the new Calibrachoa can be compared to plants of Petunia hybrida ‘Sunbelrikupi’, disclosed in U.S. Plant Pat. No. 14,729. In side-by-side comparisons, plants of the new Calibrachoa differ primarily from plants of ‘Sunbelrikupi’ in the following characteristics:
1. Plants of the new Calibrachoa are more compact than plants of ‘Sunbelrikupi’.
2. Plants of the new Calibrachoa have thicker lateral branches than plants of ‘Sunbelrikupi’.
3. Plants of the new Calibrachoa are more freely flowering than plants of ‘Sunbelrikupi’.
4. Plants of the new Calibrachoa have larger flowers than plants of ‘Sunbelrikupi’.

### BRIEF DESCRIPTION OF THE PHOTOGRAPH

The accompanying colored photograph illustrates the overall appearance of the new Calibrachoa plant showing the colors as true as it is reasonably possible to obtain in colored reproductions of this type. Colors in the photograph may differ slightly from the color values cited in the detailed botanical description which accurately describe the colors of the new Calibrachoa plant. The photograph is a side perspective view of a typical flowering plant of ‘Duealkoneo’ grown in a container.

### DETAILED BOTANICAL DESCRIPTION

The aforementioned photograph and following observations, measurements and values describe plants grown during the summer in 22-cm containers in a glass-covered greenhouse in Rheinberg, Germany and under cultural practices typical of commercial Calibrachoa production. During the production of the plants, day and night temperatures averaged 18° C. and light levels averaged 4,500 lux. Rooted young plants were pinched one time three weeks after planting and were 16 weeks old when the photograph and description were taken. In the following description, color references are made to The Royal Horticultural Society Colour Chart, 2001 Edition, except where general terms of ordinary dictionary significance are used.
Botanical classification: Calibrachoa sp. ‘Duealkoneo’.
Parentage:
   Female, or seed, parent.—Proprietary selection of Calibrachoa sp. identified as code number AA12-002806-002, not patented.
   Male, or pollen, parent.—Proprietary selection of Calibrachoa sp. identified as code number AA13-003709-002, not patented.
Propagation:
   Type.—By vegetative cuttings.
   Time to initiate roots, summer.—About five days at temperatures about 20° C.
   Time to initiate roots, winter.—About seven days at temperatures about 20° C.
   Time to produce a rooted young plant, summer.—About three weeks at temperatures about 20° C.
   Time to produce a rooted young plant, winter.—About four weeks at temperatures about 20° C.
   Root description.—Fine, fibrous; typically white in color, actual color of the roots is dependent on substrate composition, water quality, fertilizers, substrate temperature and age of roots.
   Rooting habit.—Freely branching; medium density.
Plant description:
   Plant and growth habit.—Compact, semi-upright and outwardly spreading to trailing and decumbent plant habit; freely branching habit with about five primary lateral branches developing per plant; pinching enhances branching; dense and full appearance; vigorous growth habit; moderate growth rate.
   Plant height.—About 27 cm.
   Plant diameter.—About 84 cm.
Lateral branch description:
   Length.—About 60 cm.
   Diameter.—About 7 mm.
   Internode length.—About 1.8 cm.
   Strength.—Strong.
   Aspect.—Initially upright to outwardly spreading to trailing and decumbent.
   Texture.—Pubescent.
   Color.—Close to 144B.
Leaf description:
   Arrangement.—Before flowering, alternate, and after flowering, opposite; simple.
   Length.—About 3.4 cm.
   Width.—About 1 cm.
   Shape.—Oblanceolate.
   Apex.—Rounded.
   Base.—Cuneate.
   Margin.—Entire.
   Texture and luster, upper and lower surfaces.—Pubescent; matte.
   Venation pattern.—Pinnate; arcuate.
   Color.—Developing leaves, upper surface: Close to 143A. Developing leaves, lower surface: Close to 141C. Fully expanded leaves, upper surface: Close to 137A; venation, close to 137A. Fully expanded leaves, lower surface: Close to 137C; venation, close to 137C.
   Petioles.—Length: About 2.6 mm. Diameter: About 1.6 mm. Texture, upper and lower surfaces: Pubescent. Color, upper and lower surfaces: Close to 139B.
Flower description:
   Flower arrangement and habit.—Single salverform flowers arising from leaf axils; freely flowering habit with usually about 700 to 750 flowers developing per plant during the flowering season; flowers face upright to outwardly.
   Fragrance.—None detected.
   Natural flowering season.—Early flowering habit, plants of the new Calibrachoa initiate and develop flowers about four to five weeks after planting; plants flower continuously from the spring throughout the summer in Germany.
   Flower longevity.—Individual flowers last about seven to ten days on the plant; flowers not persistent.
   Flower diameter.—About 2.5 cm.
   Flower length (height).—About 2.1 cm.
   Flower throat diameter.—About 1.1 cm.
   Flower tube length.—About 1.5 cm.
   Flower tube diameter, base.—About 1.4 mm.
   Flower buds.—Length: About 2.3 cm. Diameter: About 4.1 mm. Shape: Elongated oblong. Texture: Pubescent. Color: Close to 1A and 72A.
   Corolla.—Arrangement: Five petals fused at the base and opening into a flared trumpet. Petal length from throat: About 1.4 cm. Petal lobe width: About 1.4 cm. Petal shape: Roughly spatulate. Petal apex: Rounded. Petal margin: Entire. Petal texture and luster, upper and lower surfaces: Smooth, glabrous; matte. Throat texture: Smooth, glabrous. Tube texture: Smooth, glabrous. Color: Petal, when opening, upper surface: Close to 67A. Petal, when opening, lower surface: Close to 78A. Petal, fully opened, upper surface: Close to 74A; venation, close to 81A; with development, color becoming closer to 77A. Petal, fully opened, lower surface: Close to 78A; venation, close to 83B. Throat: Close to 79A; venation, close to 79A. Tube: Close to 1C; venation, close to 202A.
   Calyx.—Arrangement: Star-shaped calyx with five sepals; sepals fused at the base. Sepal length: About 1.2 cm. Sepal width: About 3 mm. Sepal shape: Lanceolate. Sepal apex: Acute. Sepal margin: Entire. Sepal texture and luster, upper and lower surfaces: Pubescent; matte. Color, upper surface: Close to 141B. Color, lower surface: Close to 143C.
   Peduncles.—Length: About 1.2 cm. Diameter: About 1 mm. Angle: About 45° from stem axis. Strength: Moderately strong. Texture: Pubescent. Color: Close to 143C.
   Reproductive organs.—Stamens: Quantity: Six per flower. Filament length: About 7.6 mm. Filament color: Close to 4D. Anther shape: Ellipsoidal. Anther length: About 0.8 mm. Anther color: Close to 9A. Pollen amount: Scarce. Pollen color: Close to 9A. Pistils: Quantity: One per flower. Pistil length: About 7 mm. Style length: About 7 mm. Style color: Close to 144C. Stigma shape: Ellipsoidal. Stigma color: Close to 143A. Ovary color: Close to 144D. Seeds and fruits: Seed and fruit development have not been observed on plants of the new Calibrachoa.
Garden performance: Plants of the new Calibrachoa have been observed to have good garden performance and tolerate wind, rain and temperatures ranging from about 5° C. to about 40° C.
Pathogen & pest resistance: Plants of the new Calibrachoa have not been observed to be resistant to pathogens and pests common to Calibrachoa plants.

It is claimed:

1. A new and distinct Calibrachoa plant named ‘Duealkoneo’ as illustrated and described.
